(19) United States
(12) Patent Application Publication
(10) Pub. No.: US 2025/0134680 A1
(43) Pub. Date: May 1, 2025

(54) Fabric Reinforced Liner with Fabric Covering

(71) Applicant: Alps South, LLC, St. Petersburg, FL (US)

(72) Inventors: Aldo Laghi, Pinellas Park, FL (US); David Carson, Clearwater, FL (US)

(21) Appl. No.: 18/930,718

(22) Filed: Oct. 29, 2024

Related U.S. Application Data
(60) Provisional application No. 63/546,667, filed on Oct. 31, 2023.

(51) Int. Cl. A61F 2/78; A61B 5/00

(52) U.S. Cl. CPC A61F 2/7812; A61B 5/6811

(57) ABSTRACT

A prosthetic liner having an open proximal end, a closed distal end, an inner layer comprising an elastomeric material with an inner fabric reinforcement that is bonded to an exterior fabric layer. The inner fabric reinforcement and/or the exterior fabric layer may be made of a spacer material. The liner may also comprise a distal attachment with a further fabric reinforcement and may also include a plurality of sensors distributed throughout the length of the liner. Use of the inner reinforcement fabric allows for the creation of a thinner liner than was previously possible using other known techniques.

### CROSS-REFERENCE TO RELATED APPLICATIONS

This application claim priority to U.S. Provisional Application No. 63/546,667, filed Oct. 31, 2023, titled “Fabric Reinforced Liner with Fabric Covering”, the contents of which are incorporated by reference herein in their entirety.

### FIELD OF THE INVENTION

This invention relates to liners for use in a prosthetic assembly. Specifically, the described invention relates to fabric reinforced liners with an external fabric covering.

### DESCRIPTION OF BACKGROUND ART

Prosthetic liners have been in use since the 1970's, mostly custom made, and made of various materials. Prosthetic liners serve the purpose of providing comfort for an amputee when wearing a prosthetic limb as the liner allows for a cushion between the residual limb and the socket supporting the prosthetic limb. Silicone liners have been used since the 1980s in the prosthetic industry such as those described in U.S. Pat. No. 4,923,474 granted to Klasson and Kristinsson. Other examples of such liners include U.S. Pat. No. 5,728,168 to Laghi et al., U.S. Pat. No. 5,830,237 granted to Kania, U.S. Patent No. 5,507,834 to Laghi et al., U.S. Patent No. 5,443,525 to Laghi et al., and U.S. Pat. No. 5,728,168 to Laghi et al. Gel and urethane liners have also been used for prosthetic and orthotic purposes and, for the most part, have a fabric covering. The fabric covering is used to reinforce the underlying material (silicone, gel, urethane) and allows for extensibility to make it easier to don and doff the liner by rolling it on and off the residual limb.

Elastomeric liners are preferred for prosthetics because of the comfort provided to the interface between the body and the prosthetic limb. The elasticity of the elastomeric interior helps to prevent skin breakdown and reduce the impacts of ambulation on the residual limb.

The liners disclosed in U.S. Pat. No. 5,443,525, U.S. Pat. No. 5,507,234, U.S. Pat. No. 5,728,168, U.S. Pat. No. 6,544,292, and U.S. Pat. No. 6,764,631 each represent advances in the field of fabric covered liners and the disclosures of which are hereby incorporated by reference. Additionally, U.S. Pat. No. 6,454,812, also hereby incorporated by reference, describes a liner comprising additional features attached to the liner textile to limit the vertical stretch at the distal end of a liner and is incorporated herein by reference. This method has been used successfully within the field of prosthetics to provide the liner textile with a strengthened distal end, means for attaching a threaded mechanical feature, and a boundary layer that prevents the thermoplastic elastomer from undesirably passing through the textile during high pressure processing.

In addition, liners with a distal attachment for a locking system have been used on prosthetic devices for many years. As described in U.S. Pat. No. 6,454,812 to Laghi, one of the co-inventors of the present invention and incorporated herein by reference, this system has to attach in a secure method to the distal portion of the liner to prevent separation or pistoning (the distal distraction of the liner from the residual limb). Many methods of securing a metal distal portion including adhering using adhesive materials, grommetting, and molding. This could result in locking systems that had an exposed stem that required protection by covering with silicone, gel, or other material unrelated to the locking system itself and which could cause the system as a whole to not be as secure thereby potentially causing separation or pistoning. In addition, prior art systems generally taught that distal umbrellas mounted to an external surface of the fabric of the prosthetic liner.

The primary drawbacks to the prior art are lack of strength and effort to don. Most amputees have had amputations for vascular, as opposed to traumatic, reasons. This means that the amputation was because of poor circulation. Most amputees are also elderly. As such, most amputees have thinner, more delicate, skin which is prone to damage and have reduced blood flow to the extremities giving them less ability to heal sores and wounds. Some elderly amputees end up having recurring amputations as the skin of their residual limbs becomes damaged and infected. Furthermore, there is a buildup of heat while in use based on the thickness of the elastomer layer of any liner due to the thermal capacity of the materials used. A lower weight liner allows for less energy expenditure by the amputee which allows for a more active lifestyle. Accordingly, there exists a need for an improved liner construction that has greater durability and allows for wicking away of sweat from the limb.

This present invention relates to use of an impregnated fabric layer underneath an external fabric layer to provide strength and comfort while also allowing for a thinner internal layer. A spacer fabric can be incorporated to increase comfort while in use. Sensors or sweat ports may also be introduced to help track issues for patients and to decrease issues related to sweat and other moisture.

Therefore, it is an object of this invention to provide an improvement which overcomes the aforementioned inadequacies of the prior art devices and provides an improvement which is a significant contribution to the advancement of the prosthetic liner art.

Another object of this invention is to provide a liner with greater durability and resistance to tears, cuts, and wear.

Another object of this invention is to allow the use of softer/weaker elastomers internally which provide additional comfort to the user.

Another object of this invention is to allow the use of reinforcing fabrics that wick sweat away from the limb.

Another object of this invention is to allow for impact cushioning in select regions.

Another object of this invention is to allow for easier donning and doffing by using a reinforcing fabric that has a high circumferential stretch used in combination with an elastomer having a low modulus of elasticity.

Another object of this invention is to allow the liner to be thinner and lighter than currently available prosthetic liners.

Another object of this invention is to allow for the use of sensors that are either stitched or embedded into the reinforced fabric layer, wound dressing, or pathways for sweat channeling.

Another object of this invention is to allow for the inclusion of transdermal drug delivery to improve the health of the limb.

Another object of the invention is to allow for greater customization options in terms of external fabric used as well as type of elastomer used.

Another object of the invention is to create an auxiliary compression force for the user which can provide a sense of security and comfort.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The long-standing but heretofore unfulfilled need for an apparatus that overcomes the limitations of the prior art is now met by a new, useful, and non-obvious invention. The invention meets the need for a new prosthetic liner that overcomes the issues inherent in the prior art.

The present invention relates generally to a prosthetic liner having an open proximal end, a closed distal end, an inner layer comprising an elastomeric material with an inner fabric reinforcement that is bonded to an exterior fabric layer. The inner fabric reinforcement and/or the exterior fabric layer may be made of a spacer material. The liner may also comprise a distal lock with a further fabric reinforcement. Use of the inner reinforcement fabric allows for the creation of a thinner liner than was previously possible using other known techniques.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a perspective view of the gel layer of the present invention;

FIG. 2 is a cross-sectional view of the gel layer of FIG. 1 along line I-I;

FIG. 2A shows detail J from FIG. 2;

FIG. 3 is a perspective view of the external fabric layer of the present invention;

FIG. 4 is a cross-sectional view of the external fabric layer of FIG. 3 along line K-K;

FIG. 5 is a perspective view of an alternative embodiment of the present invention;

FIG. 6 is a cross-sectional view of the embodiment shown in FIG. 5 along line L-L;

FIG. 6A show detail M from FIG. 6;

FIG. 7 is a cross-sectional view of another alternative embodiment of the present invention showing the use of spacer fabric;

FIG. 8 is a cross-sectional view of another alternative embodiment of the present invention showing spacer fabric with an additional gel layer;

FIG. 9 is a perspective view of another alternative embodiment of the present invention;

FIG. 10 is a cross-sectional view of FIG. 9 along line O-O;

FIG. 10A show detail P from FIG. 10;

FIG. 11 is a perspective view of another alternative embodiment of the present invention;

FIG. 12 is a cross-sectional view of FIG. 11 along line S-S;

FIG. 12A shows detail T of FIG. 12;

FIG. 13 is a perspective view of another alternative embodiment of the present invention;

FIG. 14 is a cross-sectional view of FIG. 13 along line T-T;

FIG. 14A shows detail V of FIG. 14;

FIG. 15 is a perspective view of another alternative embodiment of the present invention;

FIG. 16 is a cross-sectional view of FIG. 15 along line V-V;

FIG. 17 is a perspective view of another alternative embodiment of the present invention;

FIG. 18 is a cross-sectional view of FIG. 17 along line X-X;

FIG. 19 is a perspective view of another alternative embodiment of the present invention;

FIG. 20 is a cross-sectional view of FIG. 19 along line AA-AA; and

FIG. 20A shows detail Z from FIG. 20.

Similar reference numerals refer to similar parts throughout the several views of the drawings.

### DETAILED DESCRIPTION OF THE DRAWINGS

The following description is of the best mode presently contemplated for carrying out the invention. This description is not to be taken in a limiting sense, but is made merely for the purpose of describing one or more preferred embodiments of the invention. The scope of the invention should be determined with reference to the claims.

Throughout the figures, the prosthetic liner 10 of the present invention is generally cylindrical and comprises an open upper end 12 for receiving a residual limb, not shown, and a closed distal end 14 with sidewalls 16.

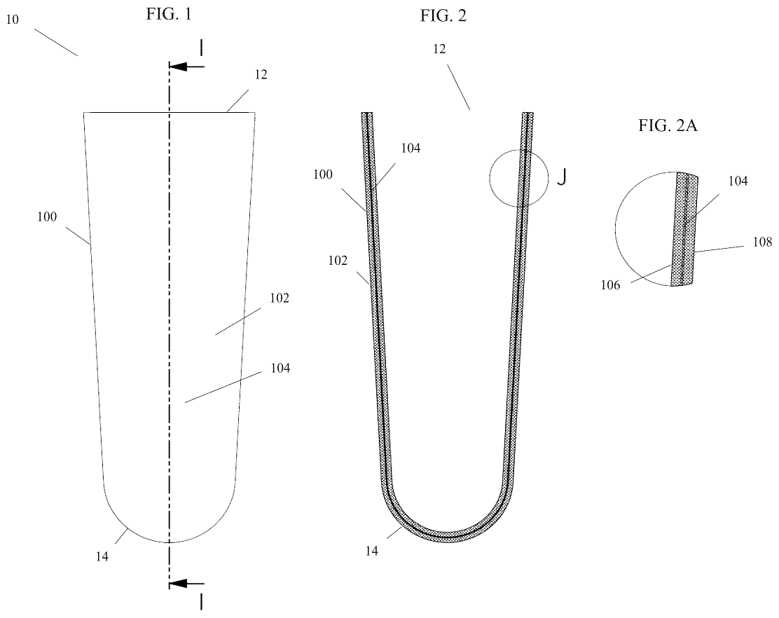

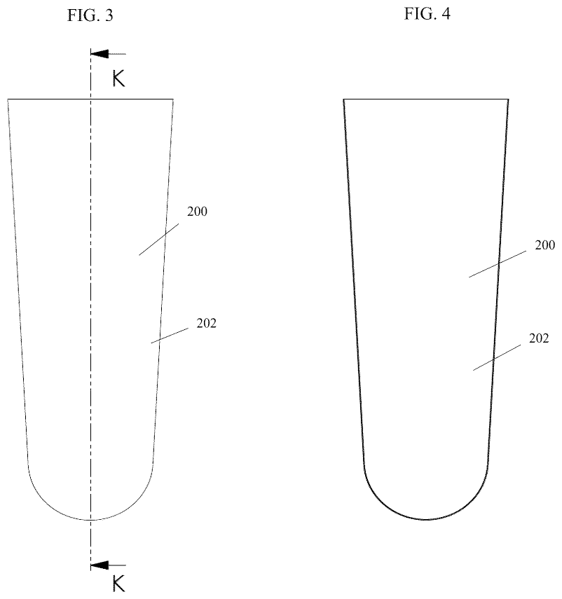

As seen in FIGS. 1 through 2A, the inner layer 100 of a preferred embodiment uses a thermoplastic material 102 shaped as the prosthetic liner 10 is described above. The thermoplastic material 102 may be a styrene-based thermoplastic elastomer, urethane, silicone, or any other known moldable material in the art now known or later discovered. The thermoplastic material also has an interior fabric 104 that is fully impregnated with the thermoplastic material 102 so that the interior fabric 104 provides reinforcement to the inner layer 100. The inner layer is preferably between 1 millimeter and 18 millimeters thick, preferably around 3 millimeters. The interior fabric 104 is preferably polyester but may be any fabric that carries a stretch when force is applied and may be woven or non-woven. This includes natural fiber cotton and lycra, spandex, or other elastic material. The interior fabric 104 is preferably centrally located within the thickness of the thermoplastic material 102 but may be located at any point such that the thermoplastic material 102 has a skin interface side 106 and a fabric interface side 108. FIGS. 3 and 4 depict the exterior layer 200 which comprises an exterior fabric 202 shaped (via knitting and stitching) as the prosthetic liner 10 is described above and generally having a thickness between 1 millimeter and 18 millimeters thick. As seen in FIG. 4, the exterior layer 200 carries no thermoplastic material 102. The exterior fabric 202 is preferably polyester but must be a fabric that carries a stretch when force is applied. The interior fabric 104 and exterior fabric 202 do not need to be the same material but interior fabric 104 must have the same stretch capabilities as exterior fabric 202 in order to prevent shearing. Shearing occurs when an exterior layer of a prosthetic liner travels further than an interior layer when the liner is in use thereby causing rubbing or other frictional forces against the skin of the user. It is important to prevent shearing because it can contribute to tissue breakdown on a residual limb.

The combination of the thermoplastic material 102 and the interior fabric 104 creates an elastomeric base with strength and tear resistance. On average, the inner layer 100 has a thickness of 0.01 to 2 millimeters thick which is thinner than average liners due to the inclusion of the interior fabric 104. The stronger composite allows for reduced effort during ambulation and/or a cooler feeling due to a reduction of thermal mass. The inclusion of the interior fabric 104 also increases the number of uses a liner may have when using a softer thermoplastic material 102 or when pursuing more vigorous activities. In addition, the interior fabric 104 will help to prevent tears or the propagation of tears, a known problem with silicone liners.

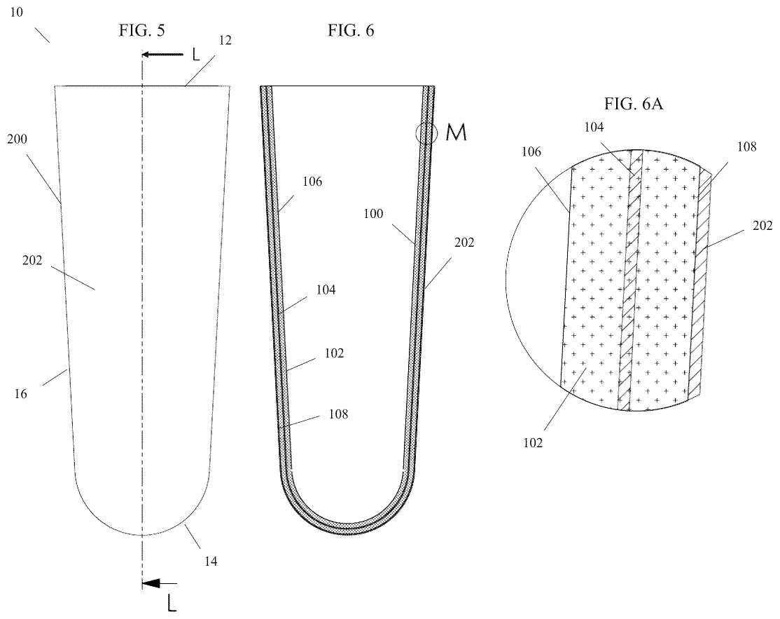

As seen in FIGS. 5-6A, the preferred embodiment of the prosthetic liner 10 has the interior layer 100 forming a composite with exterior layer 200 thereby creating multiple layers of fabric within the prosthetic liner 10. As seen in more detail in FIG. 6A, the thermoplastic material 102 permeates and impregnates the interior fabric 104 but is not present in the exterior fabric 202; instead attaching to an interior surface 204 no more than necessary to bond.

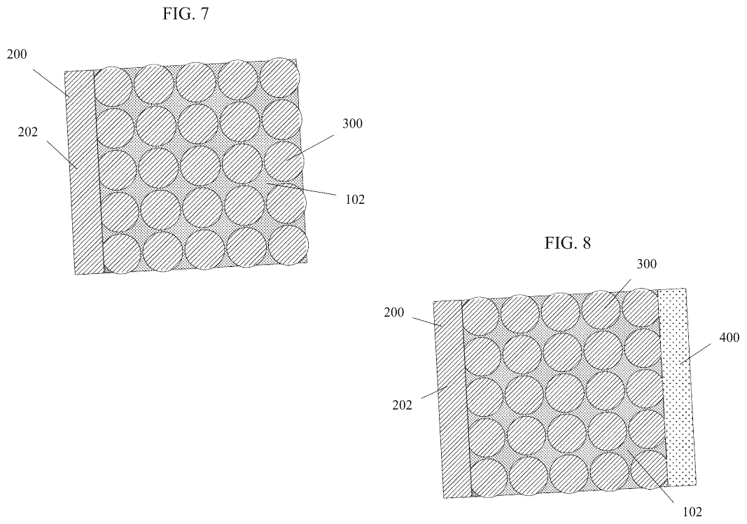

FIGS. 7 and 8 show an alternative embodiment of the present invention. Here, as shown in FIG. 7, the exterior layer 200 and exterior fabric 202 is attached to a spacer fabric 300 (as represented by the plurality of circles) which is impregnated with the thermoplastic material 102. The spacer fabric 300 may be any type of fabric wherein there are distinct top and bottom layers with interstitial support fibers. A type of spacer fabric that may be used in the present invention is described in U.S. Publication No. 2023-0277340. Alternatively, as shown in FIG. 8, the spacer fabric 300 impregnated with thermoplastic material 102 may form a composite with the exterior layer 200 by including an additional secondary interior gel layer 400 made of the same or different thermoplastic material 102 as is used in the inner layer 100.

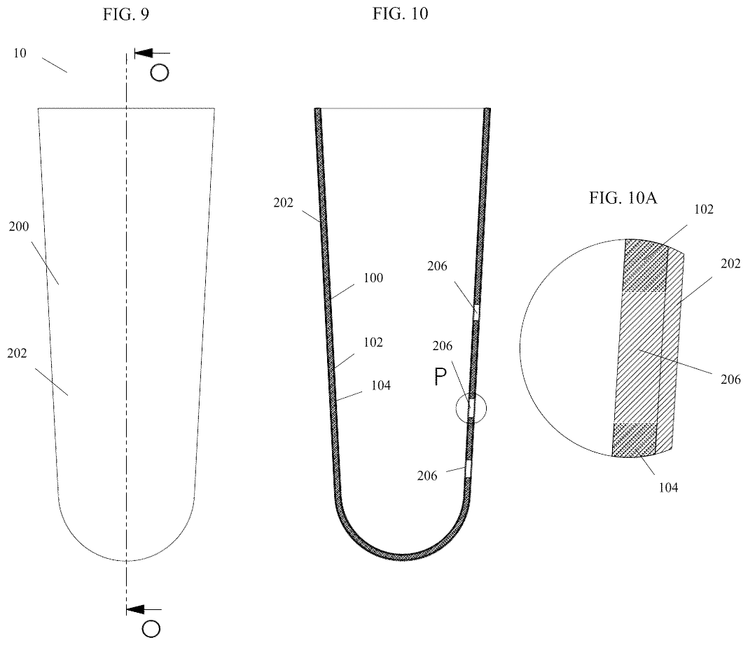

FIGS. 9-10A show another alternative embodiment. In this embodiment, the inner layer 100 includes a plurality of voids 206. In this particular embodiment, the interior fabric 104 is fully impregnated with the thermoplastic material 102, as shown in FIG. 10A. These voids allow for the wicking of sweat or other moisture that may have been introduced inside the prosthetic liner 10 while in use, particularly if the exterior layer 200 is made of a moisture wicking material. These voids 206 may be placed randomly within the inner layer 100 but are preferably located closer to the distal end 14 as that is where sweat/moisture normally accumulates due to gravity. These voids 206 are the same thickness as the inner layer 100 so that there is contact with the skin of the user. The length of the voids 206 can be between 1 millimeter and 10 millimeters.

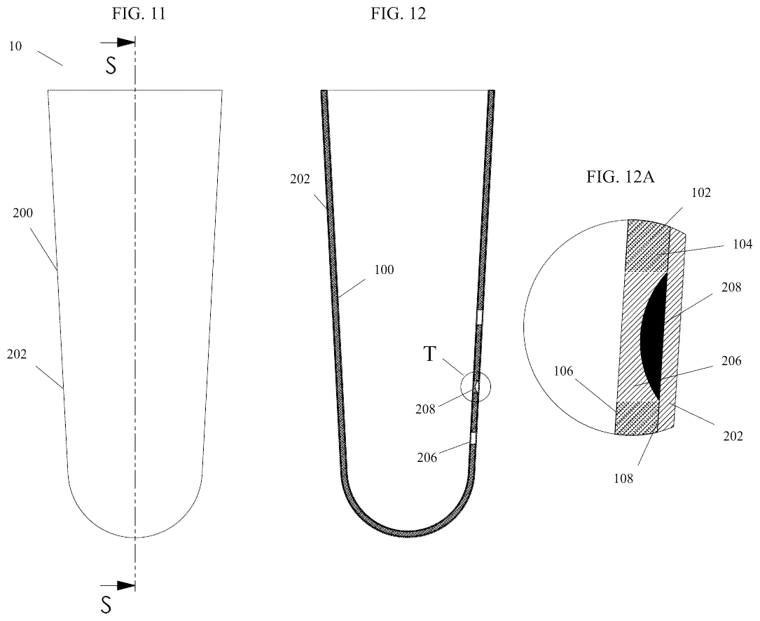

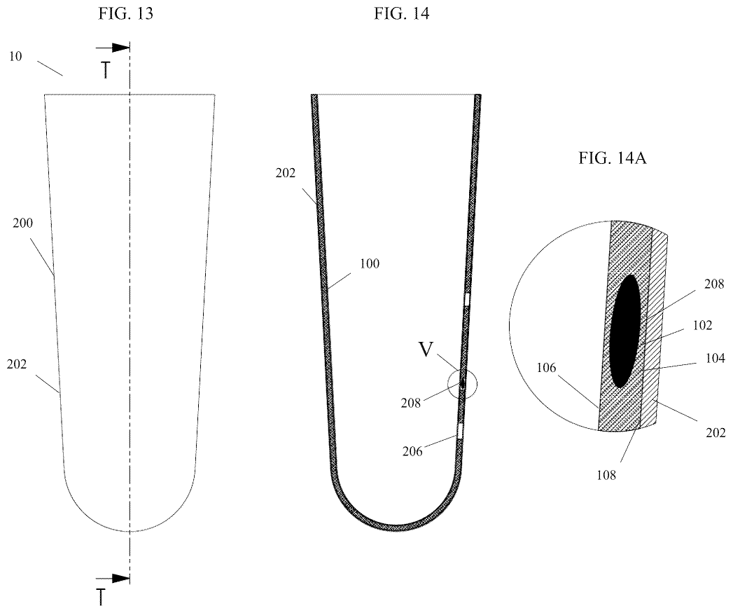

As shown in FIGS. 11-12A, another alternative embodiment may include a plurality of sensors 208 within voids 206. Much like the voids 206 themselves, the sensors 208 may be placed randomly throughout the inner layer 100 but are preferably placed in locations that allow for the sensors 208 to make adequate readings. Alternatively, as shown in FIGS. 13-14A, the sensors 208 may be molded into the thermoplastic material 102. The sensors 208 in either embodiment may be a variety of types including myoelectric control sensors, humidity moisture sensors, pressure sensors, or other sensors that provide useful information. The type of sensor 208 used and whether the sensor 208 is in a void 206 or molded into the thermoplastic material 102 is dependent on the needs of the patient.

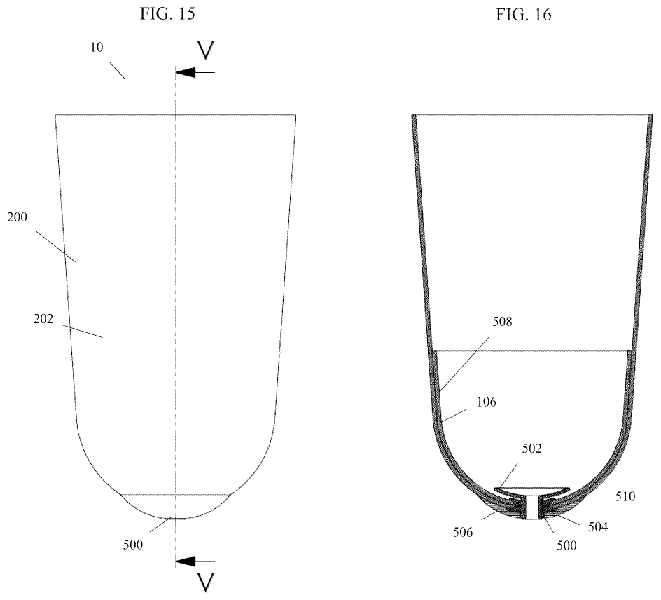

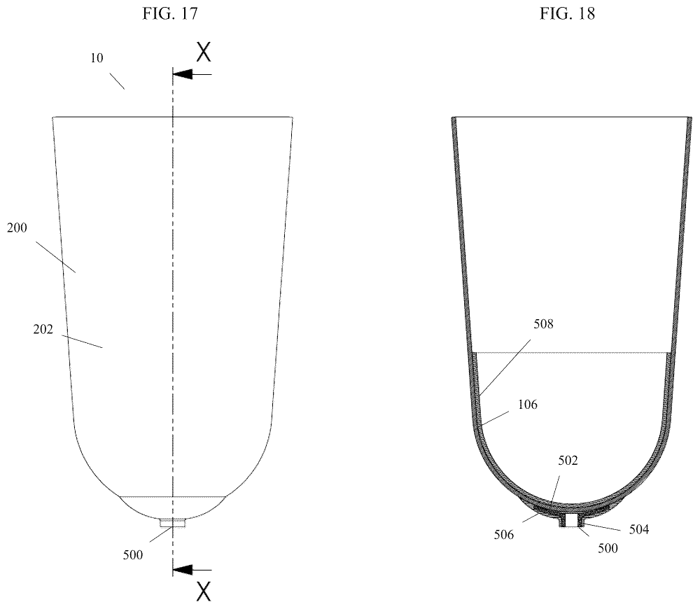

As shown in FIGS. 15-18, two alternative embodiments are shown. The first, as shown in FIG. 15-16, provides for a prosthetic liner 10 with a distal attachment 500 at the closed distal end 14. The distal attachment 500 comprises an umbrella 502, a neck 504, and encapsulation 506. In addition, a further reinforcement 508 can be provided that circumferentially lines the skin interface side 106 of the inner layer 100. The reinforcement 508 is bonded to the exterior layer 200 using urethane, silicone, or similar material. The umbrella 502 is then compressed into the reinforcement 508 and the exterior layer 200 and retained in place using a grommet 510 or similar fastener. This limits the stretch distally so as to prevent pistoning. The reinforcement 508 may be another material now known or to be discovered that stretches but also adds strength to the composite. The encapsulation 506, once molded onto the prosthetic liner 10, further locks the distal attachment 500 in place. Alternatively, as shown in FIGS. 17-18, the distal attachment 500 may be glued onto the exterior layer 200 and then have an encapsulation 506 added to keep the distal attachment 500 in place.

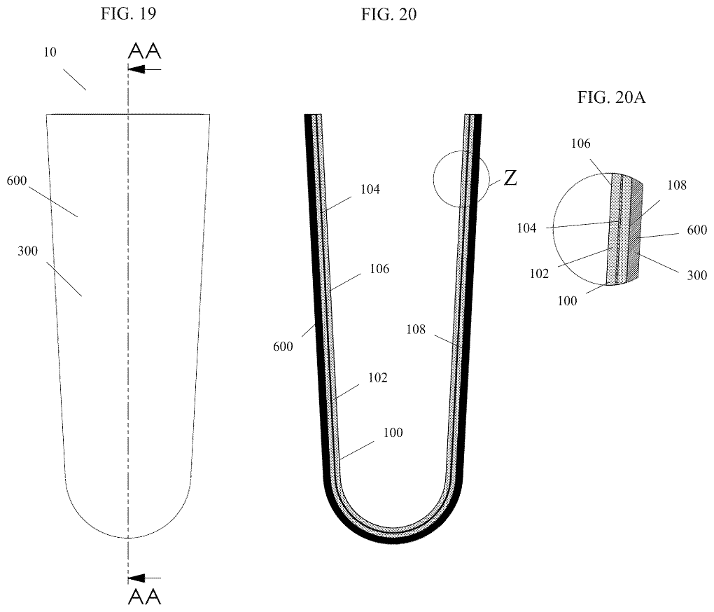

As shown in FIGS. 19-20, another alternative embodiment is shown. In this embodiment, a spacer fabric layer 600 is used as a replacement for the exterior layer 200. The spacer fabric layer 600 comprises a spacer fabric 300 with no thermoplastic material 102 present more than necessary to bond the inner layer 100 to the spacer fabric layer 600.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

1. A fabric reinforced prosthetic liner comprising:
the prosthetic liner having an open proximal end, closed distal end, and sidewalls wherein the prosthetic liner has an inner thermoplastic layer comprising a moldable material and an external fabric layer comprising a knitted fabric having a fabric thickness, the inner thermoplastic layer further comprising an interior fabric impregnated with the moldable material;
wherein the interior fabric and the knitted fabric do not exhibit shearing when stretched and the knitted fabric does not contain any moldable material through the fabric thickness.

2. The fabric reinforced prosthetic liner of claim 1 wherein the knitted fabric is polyester.

3. The fabric reinforced prosthetic liner of claim 1 further comprising at least one void containing a sensor.

4. The fabric reinforced prosthetic liner of claim 1 wherein the exterior fabric layer is a spacer fabric.

5. The fabric reinforced prosthetic liner of claim 1 wherein the thickness of the knitted fabric is between 1 millimeter and 18 millimeters.

6. e fabric reinforced prosthetic liner of claim 1 wherein the interior fabric has a thickness between 0.1 millimeters and 2 millimeters.

7. The fabric reinforced prosthetic liner of claim 1 wherein the moldable material is silicone.

8. The fabric reinforced prosthetic liner of claim 1 wherein the moldable material is a styrene-based thermoplastic elastomer.

9. A fabric reinforced prosthetic liner comprising:
the prosthetic liner having an open proximal end, closed distal end, and sidewalls wherein the sidewalls have an inner thermoplastic layer comprising a moldable material and an external fabric layer comprising a stitched fabric having a fabric thickness, the inner thermoplastic layer further comprising an interior fabric impregnated with the moldable material;
wherein the interior fabric and the knitted fabric do not exhibit shearing when stretched and the knitted fabric does not contain any thermoplastic material through the fabric thickness.

10. The fabric reinforced prosthetic liner of claim 9 wherein the stitched fabric is polyester.

11. The fabric reinforced prosthetic liner of claim 9 further comprising at least one void containing a sensor.

12. The fabric reinforced prosthetic liner of claim 9 wherein the thickness of the knitted fabric is between 1 millimeter and 18 millimeters.

13. The fabric reinforced prosthetic liner of claim 9 wherein the interior fabric has a thickness between 0.1 millimeters and 2 millimeters.

14. The fabric reinforced prosthetic liner of claim 9 wherein the moldable material is silicone.

15. The fabric reinforced prosthetic liner of claim 9 wherein the moldable material is a styrene-based thermoplastic elastomer.

16. A fabric reinforced prosthetic liner comprising:
the prosthetic liner having an open proximal end, closed distal end, and sidewalls wherein the sidewalls have an inner thermoplastic layer comprising a moldable material and an external fabric layer comprising a fabric having a fabric thickness, the inner thermoplastic layer further comprising an interior spacer fabric impregnated with the moldable material;
wherein the interior fabric and the knitted fabric do not exhibit shearing when stretched and the knitted fabric does not contain any moldable material through the fabric thickness.

17. The fabric reinforced prosthetic liner of claim 16 further comprising a secondary inner thermoplastic layer.

18. The fabric reinforced prosthetic liner of claim 16 wherein the thickness of the knitted fabric is between 1 millimeter and 18 millimeters.

19. The fabric reinforced prosthetic liner of claim 16 wherein the interior fabric has a thickness between 0.1 millimeters and 2 millimeters.

20. The fabric reinforced prosthetic liner of claim 16 further comprising at least one void containing a sensor.
